(19) United States
(12) Patent Application Publication
(10) Pub. No.: US 2013/0035581 A1
(43) Pub. Date: Feb. 7, 2013

(54) AUGMENTED REALITY ENHANCED TRIAGE SYSTEMS AND METHODS FOR EMERGENCY MEDICAL SERVICES

(71) Applicant: Guy Robert Vesto, Kildeer, IL (US)

(72) Inventor: Guy Robert Vesto, Kildeer, IL (US)

(73) Assignee: General Electric Company, Schenectady, NY (US)

(21) Appl. No.: 13/204,524

(22) Filed: Aug. 5, 2011

(51) Int. Cl. G06Q 50/00; A61B 5/0402; A61B 6/00; A61B 5/00

(52) U.S. Cl. 600/407; 600/300; 600/509; 705/3

(57) ABSTRACT

An example system includes a virtual emergency medical record (vEMR) to be dynamically generated for a patient encountered by emergency medical services personnel, the vEMR created to store and organize patient information obtained from the patient at an emergency site to be transferred to an electronic medical record for the patient at a healthcare facility. The example system includes an image recognition and search service to identify the patient based on one or more obtained images and to provide information to the vEMR. The example system includes a prior history service to search for prior clinical information for the patient and to provide information to the vEMR. The example system includes triage decision support to process patient information and prior clinical information via the vEMR to provide a) at least one of a treatment guideline and a diagnosis protocol and b) an instruction for transfer priority to a healthcare facility.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND

The present disclosure generally relates to patient emergency response coordination. More particularly, the present disclosure relates to methods, apparatus, articles of manufacture, and systems to assist in customized, dynamic emergency response coordination for a patient via a mobile platform.

If a medical emergency occurs (e.g. a car accident, heart attack, stroke, broken bone, etc.), quick action can be important to save lives and reduce permanent injury. If an ill or injured person and/or others with that person cannot obtain up-to-date care information rapidly, the lack of information can cause problems for effective treatment of the individual and potentially endanger the individual and/or delay treatment.

Sorting patients is a weak link in the emergency care system. Studies have found that triage in the emergency ambulance service at times misses patients with life-threatening conditions. Furthermore, research published in the United Kingdom-based “Emergency Medicine Journal” concluded that ambulance triage systems miss more than 50% of people who have had a stroke. Stroke is the third most common cause of death and the leading cause of severe disability.

Emergency Medical Services (EMS) first responders are forced to make triage decisions based on limited available information and often under tremendous pressure such as in mass casualty scenarios. As a consequence, many people die or are disabled from heart attack, cardiac arrest, and stroke because they do not get lifesaving treatment in time.

### BRIEF SUMMARY

Certain examples provide methods, systems, apparatus, and/or articles of manufacture for patient emergency response coordination.

Certain examples provide an emergency medical services triage system including a memory to store at least patient information, knowledge base information, and triage decision support information. The example system also includes a virtual emergency medical record to be dynamically generated for a patient encountered by emergency medical services personnel, the virtual emergency medical record created to store and organize patient information obtained from the patient by the emergency medical services personnel at an emergency site to be transferred to an electronic medical record for the patient at a healthcare facility. The example system includes a processor to process input patient information to implement an image recognition and search service, a prior history service, and triage decision support. The image recognition and search service is to identify the patient based on one or more obtained images and to provide the patient information to the virtual emergency medical record generated for the patient. The prior history service is to search for prior clinical information for the patient and to provide the prior clinical information to the virtual emergency medical record. The triage decision support is to process the input patient information and prior clinical information via the virtual emergency medical record to provide a) at least one of a treatment guideline and a diagnosis protocol and b) an instruction for transfer priority to a healthcare facility.

Certain examples provide a tangible, computer-readable storage medium including executable program instructions stored on the medium that, when executed by a programmable system, implement an emergency medical services mobile triage system. The example system includes a memory to store at least patient information, knowledge base information, and triage decision support information. The example system also includes a virtual emergency medical record to be dynamically generated for a patient encountered by emergency medical services personnel, the virtual emergency medical record created to store and organize patient information obtained from the patient by the emergency medical services personnel at an emergency site to be transferred to an electronic medical record for the patient at a healthcare facility. The example system includes an image recognition and search service to identify the patient based on one or more obtained images and to provide the patient information to the virtual emergency medical record generated for the patient. The example system includes a prior history service to search for prior clinical information for the patient and to provide the prior clinical information to the virtual emergency medical record. The example system includes triage decision support to process the input patient information and prior clinical information via the virtual emergency medical record to provide a) at least one of a treatment guideline and a diagnosis protocol and b) an instruction for transfer priority to a healthcare facility.

Certain examples provide a computer-implemented method of emergency medical services patient triage. The example method includes dynamically generating a virtual emergency medical record for a patient encountered by emergency medical services personnel at a location remote from a healthcare facility. The virtual emergency medical record is created to store and organize patient information obtained from the patient by the emergency medical services personnel at the remote location to be transferred to an electronic medical record for the patient at a healthcare facility. The example method also includes identifying, via an image recognition and search service, the patient based on one or more obtained images and providing the patient information to the virtual emergency medical record generated for the patient. The example method includes searching, via a prior history service for prior clinical information for the patient and providing the prior clinical information to the virtual emergency medical record. The example method includes processing, via triage decision support, the patient information and prior clinical information via the virtual emergency medical record to provide a) at least one of a treatment guideline and a diagnosis protocol and b) an instruction for transfer priority to a healthcare facility.

### BRIEF DESCRIPTION OF SEVERAL VIEWS OF THE DRAWINGS

FIG. 1 illustrates an interaction diagram of an example system to assist EMS first responders.

FIG. 2 depicts an example portable device to capture additional diagnostic and/or monitoring information from a patient at an emergency site remote from a healthcare facility.

FIG. 3 shows a flow diagram for a method of the triage process and how the invention assists the EMS personnel with one or more patients at an emergent scene.

FIG. 4 illustrates an example system to facilitate mobile evaluation and triage of patients at an emergency or other casualty site.

FIG. 5 is a block diagram of an example processor system that can be used to implement systems, apparatus, articles of manufacture, and methods shown in FIGS. 1-4 and described herein.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF CERTAIN EXAMPLES

Although the following discloses example methods, systems, articles of manufacture, and apparatus including, among other components, software executed on hardware, it should be noted that such methods and apparatus are merely illustrative and should not be considered as limiting. For example, it is contemplated that any or all of these hardware and software components could be embodied exclusively in hardware, exclusively in software, exclusively in firmware, or in any combination of hardware, software, and/or firmware. Accordingly, while the following describes example methods, systems, articles of manufacture, and apparatus, the examples provided are not the only way to implement such methods, systems, articles of manufacture, and apparatus.

When any of the appended claims are read to cover a purely software and/or firmware implementation, at least one of the elements is hereby expressly defined to include a tangible medium such as a memory, a digital video disc (DVD), compact disc (CD), etc. storing the software and/or firmware.

Certain examples enhance the ability of Emergency Medical Services (EMS) to triage patients and better prepare emergency departments (ED) in advance of the patients' arrival. Certain examples use “augmented reality” technology to locate prior relevant clinical data and applicable EMS protocols and guidelines. Certain examples create a “virtual emergency record” which streamlines ED workflows and enables telemedicine in emergency scenarios.

Certain examples include software services and mobile applications which EMS first responders can access via portable devices, smart phones, laptops, tablet computers, etc., connected to a wireless network or the Internet, for example.

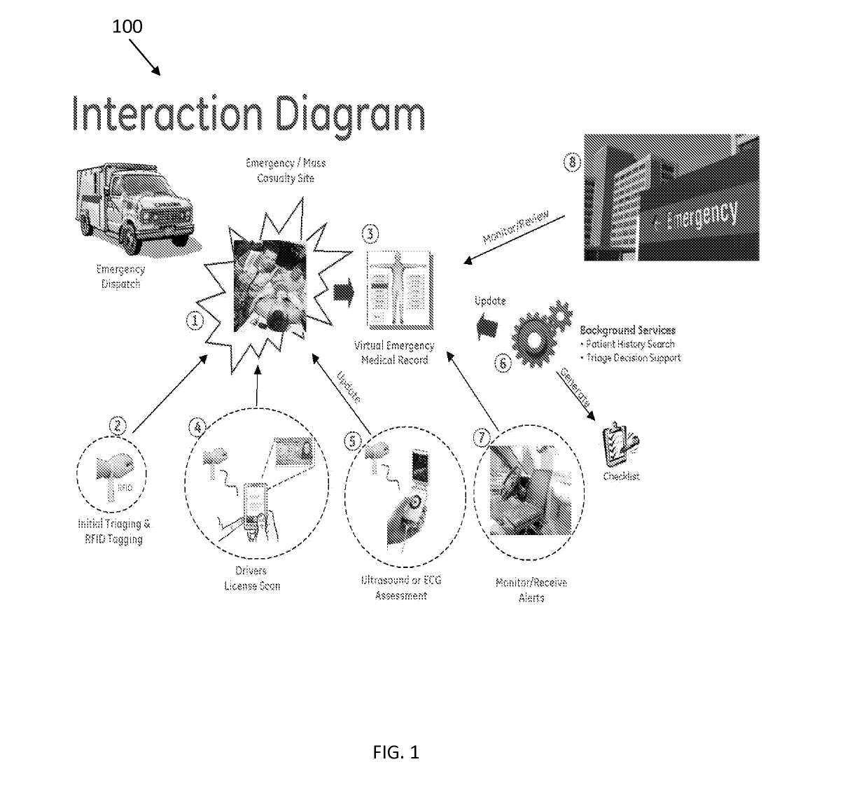

FIG. 1 illustrates an interaction diagram of an example system 100 to assist EMS first responders. As illustrated in the example of FIG. 1, first responders arrive at an emergency or casualty 1 and begin to triage patients. During the triage process, radio frequency identification (RFID) tags 2 (e.g., patient wristbands or bracelets, such as color-coded bracelets) may be used to track patients.

As the first responders move around to assess the patients, a virtual emergency medical record 3 (hereinafter vEMR) is created for each patient. A “real” emergency medical will be created later once the patients have arrived at a healthcare facility (e.g., a hospital's emergency department).

After the initial triage (or during), the EMS capture a picture or scan 4 of each patients identification (ID) card (e.g., a Driver's License, Health Insurance card, etc.), such as using an embedded camera in a smart phone or other connected portable device (e.g., a netbook, BlackBerry™, iPhone™, iPad™, Android phone, etc.). The image acquisition 4 triggers a background process 6 including, for example, image recognition and Optical Character Recognition (OCR) to identify each patient and locate prior medical history for the patient(s). The search is conducted within healthcare facility(-ies) such as hospital(s) associated with the EMS responder(s), within a Health Information Exchange (HIE) community, and/or within any connected cross community(-ies). An expanded search is to account for patients that may have traveled within the area.

If prior medical history and medical conditions are found, the EMS may be alerted 7 (depending upon the criticality). The vEMR is also updated during this process. An alert, icon, or message 7 associated with the mobile application may inform the EMS first responder if prior history is found. If so, the EMS may take pictures to document the patient's chief complaints (for example, could be certain body parts such as head, chest, stomach, leg, etc.). These additional pictures may be stored with the vEMR and forwarded to an image recognition service which determines the actual body part and locates relevant prior clinical information based on body part determination, for example. For example, if the EMS takes a picture of the patient's head, the patient's latest head computed tomography (CT) image is prefetched (if available). As another example, if the EMS takes a picture of the patient's chest, the latest chest X-Ray is prefetched (if available). The image recognition and prefetching provides the EMS with “augmented reality”.

As the EMS responder continues to assess patient(s), a paramedic may use one or more portable devices 5, such as a portable ultrasound (e.g., GE's VSCAN™ product), portable electrocardiogram (ECG), etc., to obtain additional information from a patient. Image, video, and/or other data that is captured are associated with the patient and stored in the vEMR. Emergency Department (ED) personnel, for example, may remotely monitor and review diagnostic information captured by the EMS on-site and/or in-transit to the healthcare facility 8 (e.g., a hospital's ED). Thus, the ED personnel may also assist the EMS via tele-consultation/telemedicine, for example.

In certain examples, paramedics and/or other EMS personnel are provided with applicable EMS guidelines, checklists, and/or protocols depending upon the patient's chief complaint(s). This information is assembled on the fly (e.g., in real time or substantially in real time factoring in a processing and/or communication delay) by a background service 6 and prioritized based on chief complaint(s) and patient's prior medical history.

In certain examples, in mass casualty scenarios, first responder coordinator(s) may monitor the progress and status of each patient (e.g., via an ambulance laptop computer and/or other similar device). In certain examples, an executive processor is provided to monitor multiple virtual emergency medical records as the records are being dynamically updated. The processor can provide real time (or substantially real time accounting for a system transmission and/or processing delay) status, alerts, etc., via a dashboard and/or other interface for one or more patients (e.g., to accommodate mass casualty and/or accident scenarios), for example.

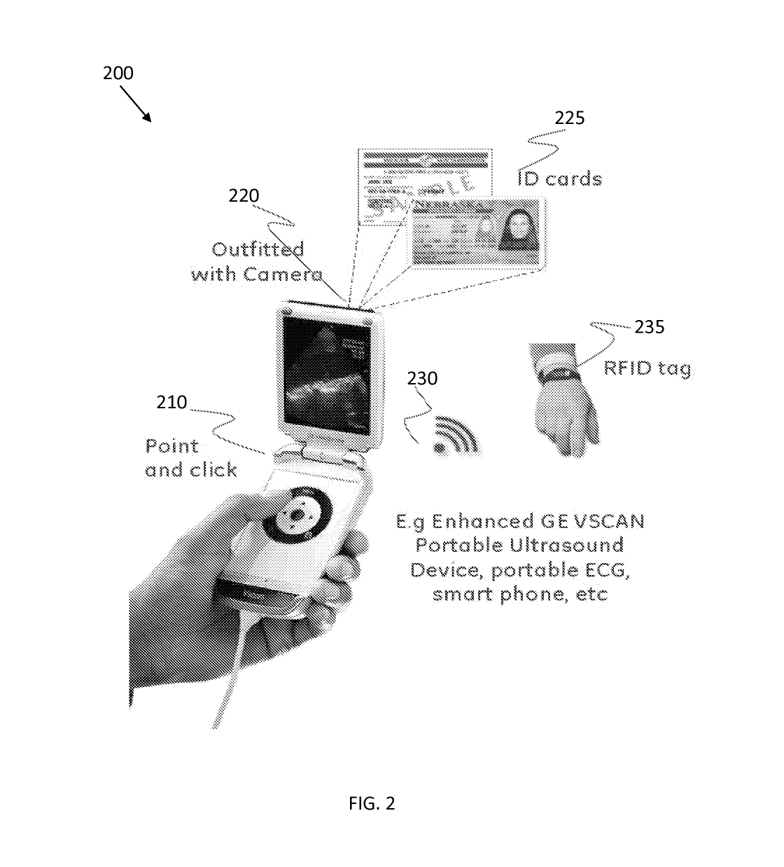

FIG. 2 depicts an example portable device 200 to capture additional diagnostic and/or monitoring information from a patient at an emergency site remote from a healthcare facility. Portable devices 200 such as ECG, ultrasound, smartphone, and/or other devices may be adapted to include mobile emergency software application(s) to assist in evaluating, triaging, and/or beginning treatment of one or more patients. For example, a portable ECG may register and store the captured ECG with the patient's vEMR. The portable device 200 of FIG. 2 illustrates how a GE Portable Ultrasound device, such as VSCAN, may be adapted for EMS use with embedded camera, RFID reader, and wireless connectivity. Such a device helps enable the EMS to use a single portable device for triaging, diagnostics, and documentation, for example.

As illustrated in the example of FIG. 2, the portable device 200 (e.g., an enhanced GE VSCAN™ Portable Ultrasound device, portable ECG device, smart phone, etc.) may be manipulated by a user to point and click 210 at a patient and/or object(s) associated with the patient. For example, using a camera 220 integrated with and/or attached to the device 200, the user may take a picture of one or more ID cards 225 associated with the patient. Additionally, an RFID receiver (or transceiver) 230 integrated with and/or attached to the device 200 may detect or be detected by an RFID tag 235 associated with the patient. The RFID tag 235 may be used to identify the patient, retrieve stored information regarding the patient, track the patient, etc.

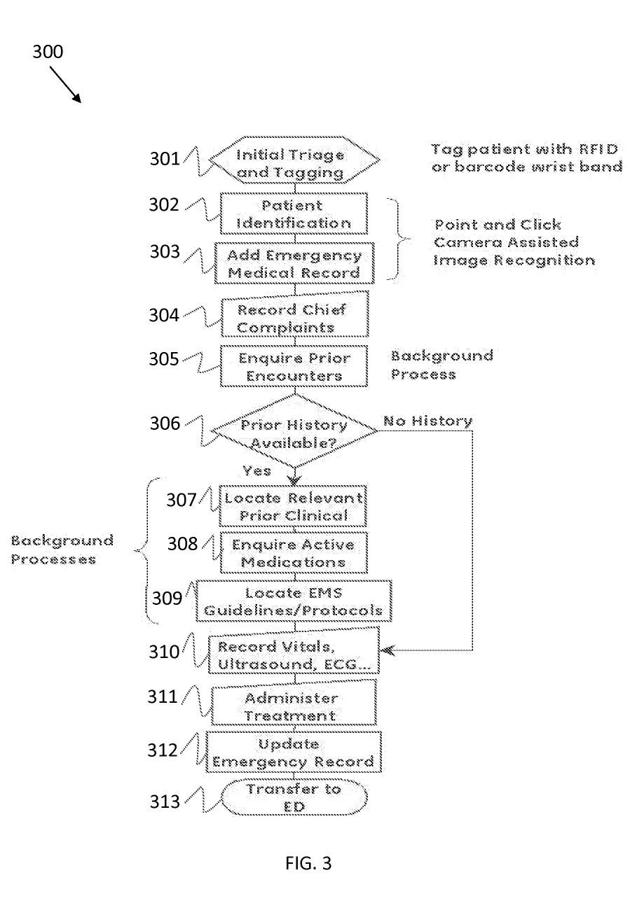

FIG. 3 shows a flow diagram for a method 300 of the triage process and how the invention assists the EMS personnel with one or more patients at an emergent scene. At block 301, an initial triage and tagging of patients is performed at the scene. For example, each patient may be tagged with an RFID and/or barcoded wrist band.

At block 302, patient identification is obtained. For example, a point and click camera photograph may be used in assisted image recognition to identify a patient. At block 303, an emergency medical record (e.g., a vEMR) is created. For example, based on the patient identification and other information gathered for the patient, the emergency medical record is created for the patient.

At block 304, chief complaint(s) for a selected patient are captured. At block 305, based on patient identification and/or other information, prior encounters involving the patient are searched. For example, while personnel are triaging patients, prior encounter and/or other patient information is queried. At block 306, if prior history is available for the patient, the prior history is analyzed. If no prior history is available, then analysis of the patient continues without the prior history information.

At block 307, if prior history is available for the patient, relevant prior clinical information is located. At block 308, active medication information may be queried for the patient. At block 309, EMS guidelines, protocols, and/or procedures, etc., may be located for the patient and prior history.

At block 310, additional information is obtained for the patient. For example, vitals, medical images (e.g., portable ultrasound), ECG, etc., are recorded. At block 311, treatment is administered to the patient. At block 312, the emergency medical record for the patient is updated. At block 313, the patient is transferred to a healthcare facility (e.g., an ED at a hospital). The emergency medical record may be transferred and/or otherwise reconciled with a patient electronic medical record and/or personal health record at the healthcare facility, for example.

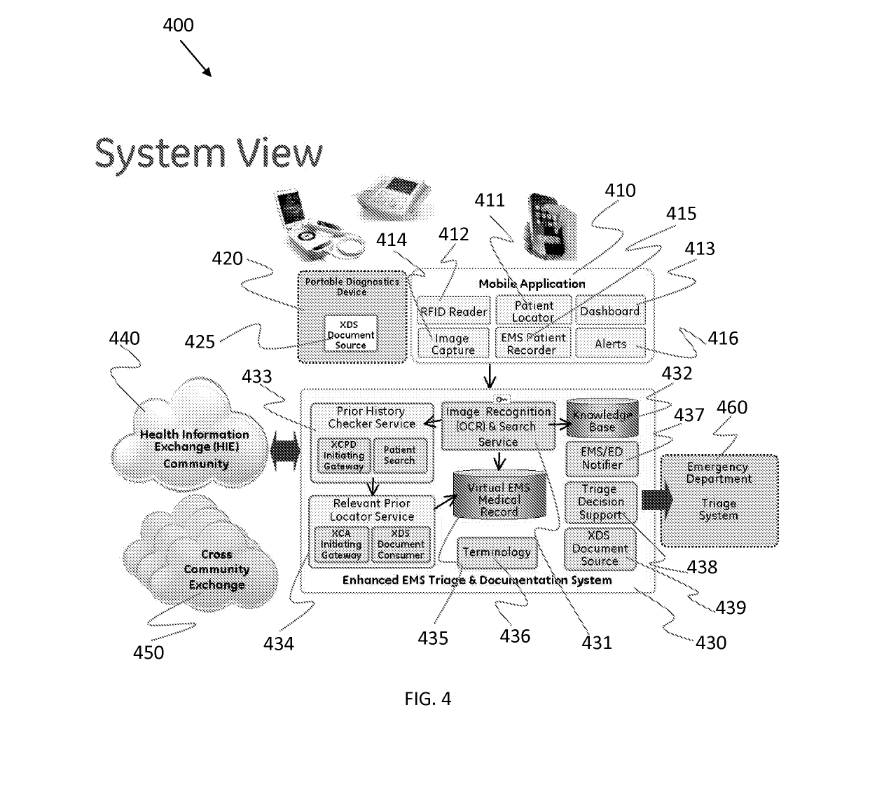

FIG. 4 illustrates an example system 400 to facilitate mobile evaluation and triage of patients at an emergency or other casualty site. The example system 400 includes a mobile application 410, a portable diagnostics device 420, and an enhanced EMS triage and documentation system 430 in communication with an HIE community 440, a cross community exchange 450, and an emergency department triage system 460, for example. Components of the system 400 may be implemented using hardware, firmware, software, and/or a combination of one or more of hardware, firmware, and software. Components of the system 400 may be implemented separately and/or in various combinations.

In certain examples, the system 400 may include and/or function in conjunction with a mobile healthcare information communication system, such as that described in U.S. patent application Ser. No. 12/189,399, assigned to General Electric Company and herein incorporated by reference in its entirety.

As shown in the example of FIG. 4, the mobile application 410 includes a patient locator 411, an RFID reader 412, a dashboard 413, an image capture module 414, an EMS patient recorder 415, and an alerts module 416. The mobile application 410 is a user facing application that interfaces with a camera 414 in the portable device and an embedded RFID reader 412. The application enable EMS first responders to record the triage process 415, locate patients (e.g., via RFID) 411, view alerts 416, update and view the vEMR, and view a dashboard 413 for the emergency site (especially useful in mass casualty scenarios, for example).

As shown in the example of FIG. 4, the portable diagnostics device 420 includes a cross-document sharing (XDS) document source 425. Portable devices such as ECG and ultrasound devices may be adapted to include and/or associate with all or part of the mobile software application(s) 410. For example, a portable ECG may register and store the captured ECG with the patient's vEMR.

As shown in the example of FIG. 4, the triage and documentation system 430 includes an image recognition (OCR) and search service 431, a knowledge base 432 (e.g., a terminology-indexed knowledge base), a prior history checker service 433, a relevant prior locator service 434, a virtual EMS or emergency medical record (e.g., vEMR) 435, a terminology 436, an EMS/ED notifier 437, a triage decision support 438, and an XDS document source 439. These services may be connected to a Health Information Exchange (HIE) and/or cross community exchanges (XCA) and hospital Emergency Room computerized triage systems, etc.

The services may use healthcare standards such as IHE actors (Integrating the Healthcare Enterprise) including Cross Community Patient Discovery (XCPD), Cross Enterprise Document Sharing (XDS), Cross Community Access (XCA), Patient Identifier Cross Reference (PIX), Clinical Document Architecture (CDA) content modules, etc.

The Triage Decision Support component 438 filters and prioritizes information forwarded to EMS personnel, which can influence the triage decision performed prior to arriving at the ED (e.g., the emergency department triage system 460).

Information provided to and/or from the mobile application 410 may include patient and/or healthcare provider information, for example. Provider information may include provider name, provider address, provider phone number, provider facsimile number, provider pager number, provider office hours, provider insurance carrier information, etc. Patient information may include, for example, one or more of the following pieces of information: patient name, patient address, patient contact information, emergency contact information, insurance information, billing information, primary care doctor information, specialist information, drug information, allergy information, current medication information, and a patient identifier. Patient information may also include patient records and reports. In addition, patient information may also include, for example, biographical information, medical history, family history, genetic test results, blood test results, heart rate, blood pressure, blood flow, and biomarker presence information. The patient identifier may be unique, for example, within a network or globally. Blood test results may include, for example, test results for blood oxygen level, white blood cell count, T-cell count, complete blood count, thyroid, cardiac risk factors, cholesterol, proteins, PSA (prostate), waste products, and glucose. In certain embodiments, patient information may come from multiple sources. For example, patient information may come from one or more of the patient, an insurance company, an in-network healthcare provider, and an out-of-network healthcare provider.

In an emergency scenario, certain examples can be used to retrieve all or part of a patient medical history and transfer the information to a healthcare provider to provide an emergency medical technician, clinician, and/or other medical staff/system with the information. In some examples, security measures, such as password and/or biometric authentication, can be built into the phone and/or retrieval device to help ensure that the individual's information was not transmitted inappropriately.

In certain examples, the HIE community 440 includes one or more patient portals, one or more physician portals, an HIE service bus, a clinical data repository, a user registry, a patient registry, a provider registry, an insurance registry, a document registry, and a document repository, for example.

In certain examples, the EMS triage and documentation system 430 exchanges data with the HIE community 440 via one or more Integrating the Healthcare Enterprise (IHE) profiles and/or frameworks, such as IHE Patient Demographics Query (PDQ), IHE Notification of Document Availability (NAV), IHE Cross Enterprise Document Sharing (XDS), and/or IHE Query for Existing Data (QED), etc. Using one or more patient and/or physician portals information from a plurality of data sources may be retrieved, stored, and/or updated via the HIE community 440. IHE profiles may be employed to provide information from the triage and documentation system 430 to the HIE community 440 and/or provide information from the HIE community 440 to the triage and documentation system 430, for example.

The following scenario of diagnosing heart attacks and understanding the best treatment further exemplifies the vulnerability in emergency care. According to the American Heart Association, STEMI (ST-Segment Elevation Myocardial Infarction) is a form of heart attack where deaths occur in the first two hours due to cardiac arrest after onset symptoms. Treatment involves administration of ASPIRIN® and admission to an operating theater or “cath lab” to have a coronary angiogram performed. A blood clot occluding the blood vessel is removed so that the narrowed coronary artery can be widened and kept dilated with a stent. Preferably, restoration of blood flow to the “at risk” myocardium should be achieved within ninety minutes.

The “Chain of survival” for STEMI patients includes the following four hurdles:
1. Early symptom recognition and call for help
2. EMS Evaluation & Treatment
3. Emergency Department Evaluation and Treatment
4. Repurfusion Therapy

According to the American Heart Association, the “Ideal Process” for Pre-hospital emergency services for STEMI patients includes:
1. Once the EMS arrive at the scene, assess airways, breathing, circulation and vital signs
2. Obtain focused history and exam
3. Acquire a twelve lead ECG as early as possible, have EMS interpret and transmit ECG and divert to PCI Center (Primary Coronary Intervention) and activate catheterization laboratory
4. Expedited transport to hospital & administer oxygen, aspirin, nitroglycerin, morphine, intravenous line en route.
5. Once the patient is at the hospital: Streamlined evaluation in ED or bypass ED, reperfusion with balloon.
   Using systems and methods described herein, certain examples improve this process and help save lives.

As another example, a training Guide for EMS Personnel states that there is no subject in the EMS field that is as little understood and, therefore, as poorly handled as mass casualty. To exemplify the complexity of mass casualties, consider the following hypothetical scenario: EMS arrives at the scene of a road traffic accident. On initial assessment, first responders notice there are two cars involved. One vehicle has caught fire with the other vehicle ten yards away with significant damage to the driver's side. All of the passengers have moved from the scene and the injured people present with the following signs and symptoms:
1.) Female, age 30, lying on the ground with visible bruising to forehead;
2.) Male, age 27, walking around with burns on his arms, crying and asking for help;
3.) Male, age 50, lying on the ground and breathing with gasping sounds;
4.) Female 20, lying on the ground with some soot around face complaining of stomach pain and asking for help in a croaky voice; and
5.) Male 30, standing next to and comforting casualty number four.

Using the “START” triage method, the paramedics on the scene triaged patient 1 as DELAYED, patients 2 and 5 as MINOR and patient 3 and 4 as IMMEDIATE. Patients 3 and 4 were immediately transported to the nearest hospital were patient 3 died after two hours. Patient 1 turned out to be six months pregnant and lost her baby a day later. Furthermore, patient 3 had a cardiac history and had stroke, which caused him to veer into oncoming traffic resulting in the accident. Both fatalities could have been avoided had the EMS first responders been aware of the patients' medical histories.

Certain examples provide a system that can be sold together with GE Healthcare devices such as the VSCAN product, any ED module part of a broader Enterprise Clinical suite, and/or as an add-on module to a Health Information Exchange. Certain examples provide methods to improve or optimize hospital ED workflows.

Certain examples provide “augmented reality” in conjunction with a clinical device and data sharing to leverage Health Information Exchanges and related standards to assist healthcare personnel in saving lives. Certain examples try to locate prior relevant clinical information and/or other potentially life critical information related to the patient in emergency scenarios. Certain examples use “augmented reality” to assist emergency workflows.

Certain examples provide a camera-assisted method for locating prior patient history and relevant information. Certain examples provide a method for building a “virtual emergency record” in the background without being intrusive to the emergency workflows and EMS first responders. Once the virtual medical record has been created, it may be continuously (or substantially continuously) updated by portable devices which can “join in” and contribute data to document the EMS workflow. Certain examples provide an ability to locate information for travelling patients using healthcare standards across healthcare communities and not be limited to a single hospital.

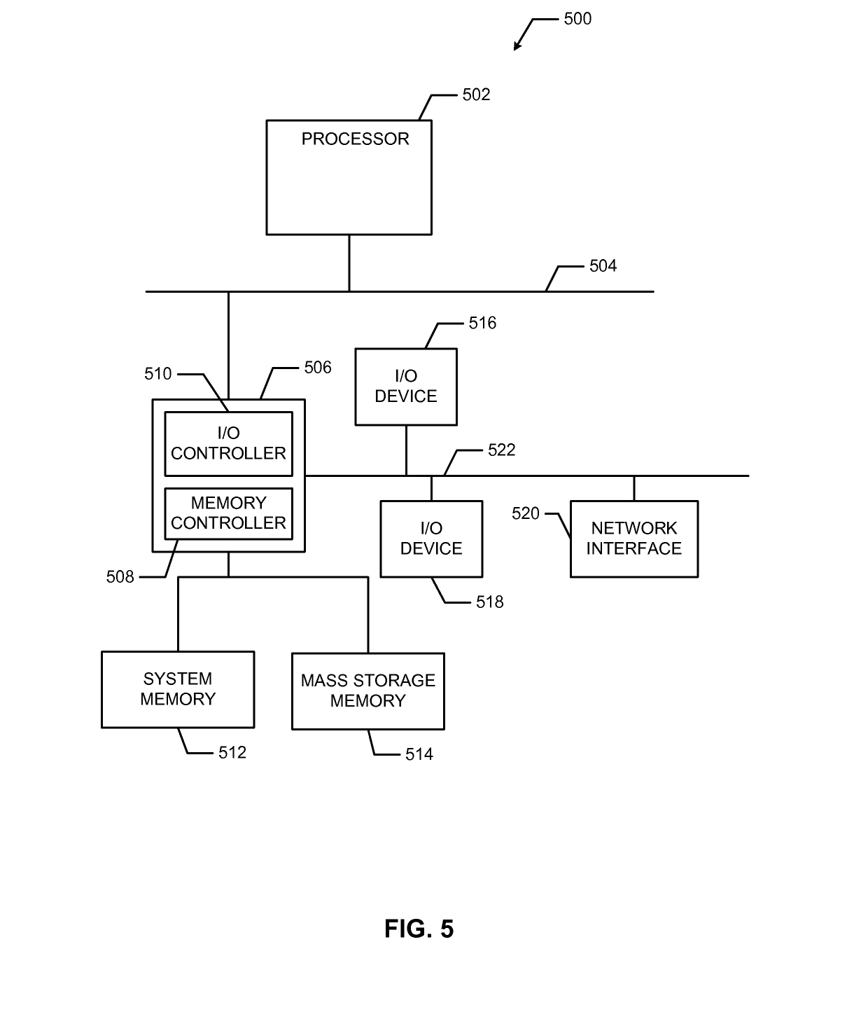

FIG. 5 is a block diagram of an example processor system 510 that may be used to implement systems, apparatus, articles of manufacture, and methods described herein. As shown in FIG. 5, the processor system 510 includes a processor 512 that is coupled to an interconnection bus 514. The processor 512 may be any suitable processor, processing unit, or microprocessor, for example. Although not shown in FIG. 5, the system 510 may be a multi-processor system and, thus, may include one or more additional processors that are identical or similar to the processor 512 and that are communicatively coupled to the interconnection bus 514.

The processor 512 of FIG. 5 is coupled to a chipset 518, which includes a memory controller 520 and an input/output (“I/O”) controller 522. As is well known, a chipset typically provides I/O and memory management functions as well as a plurality of general purpose and/or special purpose registers, timers, etc. that are accessible or used by one or more processors coupled to the chipset 518. The memory controller 520 performs functions that enable the processor 512 (or processors if there are multiple processors) to access a system memory 524 and a mass storage memory 525.

The system memory 524 may include any desired type of volatile and/or non-volatile memory such as, for example, static random access memory (SRAM), dynamic random access memory (DRAM), flash memory, read-only memory (ROM), etc. The mass storage memory 525 may include any desired type of mass storage device including hard disk drives, optical drives, tape storage devices, etc.

The I/O controller 522 performs functions that enable the processor 512 to communicate with peripheral input/output (“I/O”) devices 526 and 528 and a network interface 530 via an I/O bus 532. The I/O devices 526 and 528 may be any desired type of I/O device such as, for example, a keyboard, a video display or monitor, a mouse, etc. The network interface 530 may be, for example, an Ethernet device, an asynchronous transfer mode (“ATM”) device, an 802.11 device, a DSL modem, a cable modem, a cellular modem, etc. that enables the processor system 510 to communicate with another processor system.

While the memory controller 520 and the I/O controller 522 are depicted in FIG. 5 as separate blocks within the chipset 518, the functions performed by these blocks may be integrated within a single semiconductor circuit or may be implemented using two or more separate integrated circuits.

Thus, certain examples provide a tool for patient diagnosis and treatment. Certain examples provide a tool that can be used with a responder's existing cell phone and/or integrated with a similar device. Certain examples provide systems and methods focused on removing and/or minimizing obstacles to obtaining a highest available quality of care for a patient. Certain examples can be provided as an add-on to a clinical connectivity framework.

Certain examples contemplate methods, systems and computer program products on any machine-readable media to implement functionality described above. Certain examples can be implemented using an existing computer processor, or by a special purpose computer processor incorporated for this or another purpose or by a hardwired and/or firmware system, for example.

Some or all of the system, apparatus, and/or article of manufacture components described above, or parts thereof, can be implemented using instructions, code, and/or other software and/or firmware, etc. stored on a machine accessible or readable medium and executable by, for example, a processor system (e.g., the example processor system 510 of FIG. 5). When any of the appended claims are read to cover a purely software and/or firmware implementation, at least one of the components is hereby expressly defined to include a tangible medium such as a memory, DVD, CD, etc. storing the software and/or firmware.

FIGS. 1-4 include data and/or process flow diagrams representative of machine readable and executable instructions or processes that can be executed to implement the example systems, apparatus, and article of manufacture described herein. The example processes of FIGS. 1-4 can be performed using a processor, a controller and/or any other suitable processing device. For example, the example processes of FIGS. 1-4 can be implemented in coded instructions stored on a tangible medium such as a flash memory, a read-only memory (ROM) and/or random-access memory (RAM) associated with a processor (e.g., the processor 512 of FIG. 5). Alternatively, some or all of the example processes of FIGS. 1-4 can be implemented using any combination(s) of application specific integrated circuit(s) (ASIC(s)), programmable logic device(s) (PLD(s)), field programmable logic device(s) (FPLD(s)), discrete logic, hardware, firmware, etc. Also, some or all of the example processes of FIGS. 1-4 can be implemented manually or as any combination(s) of any of the foregoing techniques, for example, any combination of firmware, software, discrete logic and/or hardware. Further, although the example processes of FIGS. 1-4 are described with reference to the flow diagram of FIG. 3, other methods of implementing the processes of FIGS. 1-4 can be employed. For example, the order of execution of the blocks may be changed, and/or some of the blocks described may be changed, eliminated, sub-divided, or combined. Additionally, any or all of the example processes of FIGS. 1-4 can be performed sequentially and/or in parallel by, for example, separate processing threads, processors, devices, discrete logic, circuits, etc.

One or more of the components of the systems and/or blocks of the methods described above may be implemented alone or in combination in hardware, firmware, and/or as a set of instructions in software, for example. Certain examples may be provided as a set of instructions residing on a computer-readable medium, such as a memory, hard disk, DVD, or CD, for execution on a general purpose computer or other processing device. Certain examples may omit one or more of the method blocks and/or perform the blocks in a different order than the order listed. For example, some blocks may not be performed in certain embodiments of the present invention. As a further example, certain blocks may be performed in a different temporal order, including simultaneously, than listed above.

Certain examples include computer-readable media for carrying or having computer-executable instructions or data structures stored thereon. Such computer-readable media may be any available media that may be accessed by a general purpose or special purpose computer or other machine with a processor. By way of example, such computer-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of computer-executable instructions or data structures and which can be accessed by a general purpose or special purpose computer or other machine with a processor. Combinations of the above are also included within the scope of computer-readable media. Computer-executable instructions comprise, for example, instructions and data which cause a general purpose computer, special purpose computer, or special purpose processing machines to perform a certain function or group of functions.

Generally, computer-executable instructions include routines, programs, objects, components, data structures, etc., that perform particular tasks or implement particular abstract data types. Computer-executable instructions, associated data structures, and program modules represent examples of program code for executing steps of certain methods and systems disclosed herein. The particular sequence of such executable instructions or associated data structures represent examples of corresponding acts for implementing the functions described in such steps.

Examples may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Examples of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of embodiments of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated computer-readable media provide nonvolatile storage of computer-executable instructions, data structures, program modules and other data for the computer.

While the invention has been described with reference to certain embodiments/examples, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. An emergency medical services triage system comprising:
a memory to store at least patient information, knowledge base information, and triage decision support information;
a virtual emergency medical record to be dynamically generated for a patient encountered by emergency medical services personnel, the virtual emergency medical record created to store and organize patient information obtained from the patient by the emergency medical services personnel at an emergency site to be transferred to an electronic medical record for the patient at a healthcare facility; and
a processor to process input patient information to implement:
an image recognition and search service to identify the patient based on one or more obtained images and to provide the patient information to the virtual emergency medical record generated for the patient;
a prior history service to search for prior clinical information for the patient and to provide the prior clinical information to the virtual emergency medical record; and
a triage decision support to process the input patient information and prior clinical information via the virtual emergency medical record to provide a) at least one of a treatment guideline and a diagnosis protocol and b) an instruction for transfer priority to a healthcare facility.

2. The system of claim 1, further comprising a portable diagnostic device to obtain at least one of diagnostic and monitoring information from the patient at an emergency site remote from a healthcare facility.

3. The system of claim 2, wherein the portable diagnostic device comprises a smart phone including mobile emergency software to assist in evaluating, triaging, and beginning treatment of the patient.

4. The system of claim 2, wherein the portable diagnostic device comprises at least one of a portable imaging system or a portable electrocardiogram monitor.

5. The system of claim 1, wherein the processor is to communicate with a mobile application to receive location input and alerts with respect to the patient.

6. The system of claim 1, wherein the processor is to communicate with a triage system at a healthcare facility to provide information and priority regarding the patient to the healthcare facility prior to arrival of the patient at the healthcare facility.

7. The system of claim 1, wherein the prior history service is to communicate with a healthcare information exchange regarding prior clinical information for the patient.

8. The system of claim 1, wherein the prior history service is to communicate with a cross-community exchange regarding prior clinical information for the patient.

9. The system of claim 1, further comprising a dashboard to monitor multiple virtual emergency medical records and to provide dynamic status updates for a plurality of monitored patients.

10. A tangible, computer-readable storage medium including executable program instructions stored on the medium that, when executed by a programmable system, implement an emergency medical services mobile triage system comprising:
a memory to store at least patient information, knowledge base information, and triage decision support information;
a virtual emergency medical record to be dynamically generated for a patient encountered by emergency medical services personnel, the virtual emergency medical record created to store and organize patient information obtained from the patient by the emergency medical services personnel at an emergency site to be transferred to an electronic medical record for the patient at a healthcare facility;
an image recognition and search service to identify the patient based on one or more obtained images and to provide the patient information to the virtual emergency medical record generated for the patient;
a prior history service to search for prior clinical information for the patient and to provide the prior clinical information to the virtual emergency medical record; and
a triage decision support to process the patient information and prior clinical information via the virtual emergency medical record to provide a) at least one of a treatment guideline and a diagnosis protocol and b) an instruction for transfer priority to a healthcare facility.

11. The tangible, computer-readable storage medium of claim 10, further comprising a mobile application to communicate with a portable diagnostic device to obtain at least one of diagnostic and monitoring information from the patient at an emergency site remote from a healthcare facility.

12. The tangible, computer-readable storage medium of claim 11, wherein the mobile application is to receive and communicate location input and alerts with respect to the patient.

13. The tangible, computer-readable storage medium of claim 11, wherein the portable diagnostic device comprises a smart phone including mobile emergency software to assist in evaluating, triaging, and beginning treatment of the patient.

14. The tangible, computer-readable storage medium of claim 11, wherein the portable diagnostic device comprises at least one of a portable imaging system or a portable electrocardiogram monitor.

15. The tangible, computer-readable storage medium of claim 10, wherein the triage decision support is to communicate with a triage system at a healthcare facility to provide information and priority regarding the patient to the healthcare facility prior to arrival of the patient at the healthcare facility.

16. The tangible, computer-readable storage medium of claim 10, wherein the prior history service is to communicate with a healthcare information exchange regarding prior clinical information for the patient.

17. The tangible, computer-readable storage medium of claim 10, wherein the prior history service is to communicate with a cross-community exchange regarding prior clinical information for the patient.

18. A computer-implemented method of emergency medical services patient triage, the method comprising:
dynamically generating a virtual emergency medical record for a patient encountered by emergency medical services personnel at a location remote from a healthcare facility, the virtual emergency medical record created to store and organize patient information obtained from the patient by the emergency medical services personnel at the remote location to be transferred to an electronic medical record for the patient at a healthcare facility;
identifying, via an image recognition and search service, the patient based on one or more obtained images and providing the patient information to the virtual emergency medical record generated for the patient;
searching, via a prior history service for prior clinical information for the patient and providing the prior clinical information to the virtual emergency medical record; and
processing, via triage decision support, the patient information and prior clinical information via the virtual emergency medical record to provide a) at least one of a treatment guideline and a diagnosis protocol and b) an instruction for transfer priority to a healthcare facility.

19. The method of claim 18, further comprising obtaining, via a portable diagnostic device, at least one of diagnostic and monitoring information from the patient at the remote location.

20. The method of claim 18, further comprising receiving and communicating location input and alerts with respect to the patient.

21. The method of claim 18, further comprising communicating with a triage system at a healthcare facility to provide information and priority regarding the patient to the healthcare facility prior to arrival of the patient at the healthcare facility.

22. The method of claim 18, further comprising communicating with a healthcare information exchange regarding prior clinical information for the patient.

23. The method of claim 18, further comprising communicating with a cross-community exchange regarding prior clinical information for the patient.

24. The method of claim 18, further comprising monitoring multiple virtual emergency medical records and providing dynamic status updates for a plurality of monitored patients.
